(19) United States
(12) Patent Application Publication
(10) Pub. No.: US 2005/0026958 A1
(43) Pub. Date: Feb. 3, 2005

(54) Methods of therapeutic treatment using amounts of retinoid components

(75) Inventors: John Sefton, Trabuco Canyon, CA (US); John R. Gibson, Laguna Beach, CA (US)

Correspondence Address: Carlos A. Fisher;ALLERGAN, INC., T2-7H, 2525 Dupont Drive, Irvine, CA, 92612, US

(73) Assignee: Allergan, Inc.

(21) Appl. No.: 10/902,719

(22) Filed: Jul. 29, 2004

Related U.S. Application Data
(60) Provisional application No. 60/491,143, filed on Jul. 30, 2003.

(51) Int. Cl. A61K 31/4436; A61K 9/64

(52) U.S. Cl. 514/337; 424/456

(57) ABSTRACT

The invention concerns methods and compositions related to the use of a retinoid for the treatment of nodulocystic acne.

This application clams priority of U.S. Provisional Application Ser. No. 60/491,143, filed Jul. 30, 2003, which is incorporated by reference as part of this specification in its entirety.

The present invention relates to methods of providing therapeutic effects using retinoid components. More particularly, this invention relates to systemically administering to patients, that is humans or animals, without regard to the body weights of the patients, amounts of certain retinoids effective to provide reduction in the severity of various medical conditions, while, at the same time achieving one or more of consistent bioavailability, reduced drug interactions, and reduced side effects relative to administering a reference retinoid agent effective to provide the same therapeutic effect. In a preferred and more specific embodiment, the invention relates to orally administering to patients retinoid components selected from the group consisting of tazarotene, tazarotenic acid, prodrugs of tazarotenic acid, and mixtures thereof in therapeutically effective amounts, for example amounts effective to reduce conditions such as psoriasis and nodulocystic acne, advantageously while resulting in one or more of the aforementioned advantages relative to a reference retinoid agent.

Retinoid drugs exert their therapeutic activity by acting as ligands, and therefore stimulating, activating blocking or inhibiting the biological activities, of either or both of the retinoid-associated nuclear receptors RAR (retinoic acid receptors) and RXR (retinoid X receptors). Although not wishing to be limited by any particular theory, each of these receptors is thought to undergo a conformational change when a cognitive agonist binds the receptor. This conformational change then results in the receptor stimulating or inhibiting the expression of a set of particular genes. RXR and RAR appear to bind separate sets of genes. This process is termed transactivation. In addition, there are myriad ligand-mediated effects, such as involvement in the stimulation or mediation of cellular phosphorylation cascades, which may not be transactivational events.

Also, the RAR and RXR receptors each have three major subtypes. RAR receptors comprise RAR alpha, RAR beta, and RAR gamma. Similarly, RXR receptors comprise RXR alpha, RXR beta, and RXR gamma. Selective stimulation or inhibition of one or more subtype from among these 6 receptors can have different biological and therapeutic effects.

A number of retinoid drugs are formulated for oral delivery. For instance, RAR agonists such as acitretin (Soriatane®) and etretinate can be administered orally to treat psoriasis. RXR agonists such as bexarotene (Tagretin®) can be administered orally to treat skin lymphoma. Tretinoin (Vesanoid®), which binds and transactivates both RAR and RXR, can be administered orally to treat promoclocytic anemia, and isotretinoin (Accutane®), which also affect both types of receptors, can be administered orally to treat acne.

Drugs such as tretinoin and isotretinoin that affect both RAR and RXR receptors are associated with both RAR and RXR-type side effects.

Retinoids are often formulated for topical administration to be therapeutically effective while reducing the occurrence and/or severity of side effects caused by systemic administration. Topical administration of retinoids results in reduced blood concentrations of the active drug, which can adversely impact the therapeutic effectiveness of the drug. For example, the maximum blood concentration of tazarotenic acid obtained by topical administration of tazarotene is less than about 30 ng/ml. Tazarotene is currently marketed as a topical gel or cream under the brand names Tazorac® and Zorac®.

In its most basic form acne is a condition in which hair follicles, which usually permit the draining of sebum to the surface of the skin, become blocked. Due to this blockage, sebum secretion becomes blocked and bacteria begin to grow. This can lead to the formation of “whiteheads” (in which the follicle or sebaceous gland becomes totally blocked) and “blackheads” (in which the trapped sebum and bacteria partially open to the surface and turn black due to oxidation of melanin in the surface layer of the skin).

Acne occurs in different types and degrees of severity. Different types of acne include acne vulgaris, acne rosacea, acne conglobata, acne fulminans, pyroderma facialae and Gram-negative folliculitis. Mild to moderate acne may involve the appearance of whiteheads and or blackheads and can be treated by gentle cleansing and topical treatment with agents such as benzoyl peroxide. However such treatments are not generally effective in severe forms of acne such as those that involve large lesions which can result in scarring. In severe nodulocysic acne, these lesions can involve the formation of nodules (large hard bumps under the skin) or cysts (which are similar to nodules, but are pus-filled). Although not life-threatening, nodulocystic acne can cause extreme and disfiguring scarring.

Treatment for severe acne has included the oral administration of steroids and antibiotics. Isotretinoin (Accutane®) has also been used with success to treat severe nodulocystic acne, although it has a number of undesirable side effects, including teratogenic effects. Its mode of action has been thought to be due at least in part to the inhibition of sebum secretion. This reduction in sebum secretion, has been thought to be associated with the therapeutic effects of isotetinoin, see e.g., Accutane Product Monograph, revised May 15, 2003). Indeed, a number if published reports have linked the efficacy of isotretinoin in treating acne to the inhibition of sebaceous gland secretion. In particular, such reports have concluded that the marked inhibitory effect of isotretinoin on the rate of sebum secretion, for example, of about 90%, is the main factor in the clinical response of severe acne with isotretinoin. See e.g., Geiger, J. M.; Retinoids and Sebaceous Gland Activity, Dermatology, vol. 191, pps. 305-310 (1995); and Geiger, J. M. et al, Oral 13-cis Retinoic Acid is Superior to 9-cis Retinoic Acid in Sebosuppression in Human Beings, J. Am. Acad. Dermatology, vol. 34, pps. 513-515 (1996).

This reduction of sebum secretion can be detrimental to the condition of the skin. Sebum is thought to provide a natural conditioning effect, keeping the skin smooth and supple and protecting against drying, scaling and itching. Patients using isotretinoin have reported chapped lips and dry, itchy skin. Such side effects can lead to a reduction in patent compliance in the therapeutic regimen, thereby limiting the number of patients who will benefit from oral retinoid treatment for nodulocystic acne.

Chandraratna U.S. Pat. No. 5,089,509 (the disclosure of which is incorporated in its entirety herein by reference) discloses a group of compounds which may be used to treat acne and other dermatoses such as Darier's disease, psoriasis, icthyosis, eczema, atopic dermatitis and epithelial cancers, as well as in treating arthritic diseases and other immunological disorders (e.g., lupus erythematosus), in promoting wound healing, in treating dry eye syndrome and in reversing the effects of sun damage to skin. Among the compounds disclosed by Chandraratna are the compounds known as tazarotene and tazarotenic acid. The patent discloses that when the retinoid-like compounds are used in the treatment of dermatoses, it will generally be preferred to administer the drug topically, though in certain cases such as treatment of severe cystic acne, oral administration may also be used.

Firestone et al., U.S. Pat. No. 6,248,354, (the “'354 patent”) the disclosure of which is incorporated in its entirety herein by reference, discloses a capsule system for the oral delivery of an active agent, e.g., tazarotene, having low aqueous solubility and a vehicle for eliminating any need for initial active agent dissolution within the gastro-intestinal tract. The '354 patent discloses that orally administered tazarotene to provide maximum blood level concentrations of tazarotenic acid in healthy subjects of between 5.24 and 44.3 ng/ml may be sufficient to effect the treatment of acne in a patient.

Neither the Chandraratna patent nor the Firestone et al patent specifically disclose the advantages of any of the disclosed compounds, such as tazarotene and tazarotenic acid, in reducing nodular, cystic, or nodulocystic acne, for example, to obtain specific therapeutic reductions in nodulocystic acne, e.g., halting or arresting or inhibiting the progression of cystic acne, reducing or substantially eliminating one or more of the symptoms of nodulocystic acne, reducing the size of one or more lesion, reducing the number of the cystic acne lesions, substantial or complete elimination of cystic acne lesions, and the like.

Moreover, neither of these patents discloses the advantages of using such compounds at specific blood concentrations and/or for specific periods of time. Further, neither patent specifically discloses the advantages of using such compounds, for example, in reducing severe acne, such as cystic acne, at specific daily doses in specific dosage forms.

It would be advantageous to provide methods of administering retinoids to patients in amounts effective to provide therapeutic reductions in severe acne, such as nodular, cystic, or nodulosystic acne, while at the same time have less of an effect upon, for example, sebum secretion as compared to known systemically administered retinoids such as isotretinoin.

### SUMMARY OF THE INVENTION

New therapeutic methods employing retinoid components have been discovered. The present methods involve systemic, preferably oral, administration to a human or animal of a retinoid component to provide a desired therapeutic effect.

The present methods are useful in providing desired therapeutic effects, including, without limitation, the treatment, preferably the reduction of the extent of skin surface affected by pre-existing severe nodulocystic acne and the prevention of nodulocystic acne from becoming severe or widespread.

In general, the present invention is directed to methods for providing desired therapeutic effects to a human or animal which comprise systemically, preferably orally, administering to the human or animal a therapeutically effective amount of a retinoid component selected from active retinoid agents, precursors of active retinoid agents and mixtures thereof. The desired therapeutic effect advantageously is provided as a result of the administering step. In one particularly useful embodiment, the administering is effective to provide for a maximum blood concentration in the human or animal of greater than about 40 ng/ml, greater than 45 ng/ml, or greater than about 50 ng/ml.

In a particularly useful embodiment, the invention comprises new methods for treating nodulocystic acne employing retinoid components. Preferably, the retinoid component comprises tazarotene, tazarotenic acid, and prodrugs of tazarotenic acid (hereinafter collectively referred to as a “tazarotene compound”). The present methods involve systemic, preferably oral, administration to a human or animal having nodulocystic acne of such a retinoid component to provide the desired therapeutic effect, e.g., a reduction in nodulocystic acne, such as halting or arresting or inhibiting the progression of cystic acne, reducing or substantially eliminating one or more of the symptoms of cystic acne, reducing the size of one or more of the cysts, reducing the number of the cysts, substantial or complete curing of the severe cystic acne and the like.

Importantly, the currently disclosed agents, such as a tazarotene compound, do not significantly reduce sebum secretion, as is observed with currently known oral retinoids, such as Accutane® (isotretinoin). A substantial reduction in sebum secretion often renders the patient's skin dry, itchy and irritated, thereby significantly contributing to patient discomfort. Indeed, these effects may reduce the degree of patient compliance with this medication.

While it has been heretofore believed that a reduction of sebum secretion was necessary or advantageous in the treatment of severe cystic acne, the present inventors have discovered that severe nodulocystic acne may be successfully treated with less of an effect on the rate of sebum secretion than is seen in a dosage of known therapeutic retinoids, such as isotretinoin, which provides substantially similar efficacy against nodulocystic acne.

Among the advantages of reducing, or eliminating, the inhibitory effect on sebum secretion, in accordance with the present invention, are reduced incidences of dry skin (xerosis), scaling (desquamation) and itching relative to using other retinoids, such as isotretinoin, to treat nodulocystic acne with substantially equivalent efficacy. Moreover, the present methods of effectively treating nodulocystic acne with a reduced effect on sebum secretion are quite unexpected, both in view of commonly held assumption that such a reduction is an important requirement in the treatment of nodulocystic acne, and in the prior use of isotretinoin to treat severe acne.

In another aspect, the present invention is directed to methods for reducing, for example, as described elsewhere herein, nodulocystic acne, such as severe nodulocystic acne, in a human or animal which comprise systemically, preferably orally or transdermally administering to the human or animal having symptoms of nodulocystic acne a therapeutically effective amount of tazarotene. In a preferred embodiment administration performed in such a manner so as to provide a maximum blood, i.e. plasma, concentration of an active retinoid agent in the human or animal of greater than 30 ng/ml or greater than 40 ng/ml or greater than 45 ng/ml or greater than about 50 ng/ml, and more preferably greater than about 60 ng/ml or greater than about 80 ng/ml or greater than about 100 ng/ml. The desired therapeutic effect, e.g., a reduction in the symptoms, extent, and/or number of symptoms of nodulocystic acne, for example, as described elsewhere herein, advantageously is provided as a result of the administering step.

As used herein, the term “prodrug” refers to a compound or other substance which is capable of becoming converted in vivo to the compound or other substance of which it is a prodrug. For example, prodrugs of tazarotenic acid may include esters, salts, and the like which are capble of being converted to tazarotenic acid in the GI tract, the blood, the skin, or in another tissue of the body. Such conversion may be by hydrolysis, metabolization, formation, or chemical reaction, for example, after being ingested or introduced into a body of a human or animal.

For example, tazarotene and one or more derivatives of tazarotene can be considered precursors of active retinoid agents because tazarotene and one or more of its derivatives, after ingestion or introduction into the body of a human or animal, are converted into tazarotenic acid, an active retinoid agent, or one or more derivatives of tazarotenic acid, also active retinoid agents. In certain cases, a derivative of an active retinoid agent may be a precursor of an active retinoid agent and/or vice versa.

In a preferred aspect of the invention, the systemically administering step results in or is conducted at conditions effective to provide less attenuation of sebum secretion in the human or animal relative to employing a reference retinoid agent. The reference retinoid agent preferably is selected from pan RAR-active retinoids such as isotretinoin, and RXR-active retinoids, for example, bexarotene and the like. The systemically administering step using an amount of one of the reference retinoid agent is effective to provide the same reduction, for example, as described elsewhere herein, in nodulocystic acne as the present systemically administering step.

In another aspect of the present invention, the retinoid component is selected from active RAR agonists which are substantially ineffective to bind to or activate RXRs, precursors of active agonists which are substantially ineffective in binding to or activating RXRs and mixtures thereof. The systemically administering step of the present methods is effective in providing the desired therapeutic effect, and results in or is conducted at conditions effective to provide less reduction in sebum secretion in the human or animal relative to employing a RXR active retinoid agent which is effective in binding to RXRs in place of the retinoid component in a systemically administering step at a dose of the RXR active agent, or using an amount of the RXR active agent, effective to provide the same therapeutic effect, for example, the same reduction in the nodulocystic acne.

In a further aspect of the present invention, the retinoid component is selected from active RAR agonists effective to selectively, or even specifically, affect, for example, activate, at least one, and preferably both, of RAR-beta and RAR-gamma relative to RAR-alpha, precursors of such active RAR agonists and mixtures thereof. As used in this context, the term “selectively” means that the presently useful RAR agonists precursors of RAR agonists and mixtures thereof are more effective, preferably at least about 10 or about 100 times to about 1000 times or more as effective, to affect times at least one, and preferably both, of RAR-beta and RAR-gamma relative to RAR-alpha. The systemically administering step is effective to provide the desired therapeutic effect, e.g., a reduction in the nodulocystic acne, and is conducted at conditions effective to result in or to provide less reduction in sebum secretion in the human or animal relative to employing a pan active or substantially non-selective RAR retinoid agent, such as described elsewhere herein, in place of the retinoid component in a systemically administering step using an amount of the pan active or substantially non-selective RAR retinoid agent to provide the same therapeutic effect, that is the same reduction in the nodulocystic acne.

The present methods advantageously provide substantial reductions, as described elsewhere herein, in nodulocystic acne. Preferably, nodulocystic acne reductions of at least about 60% or at least about 70% or at least about 80% or at least about 85% or at least about 90% are provided, with less reduction in sebum secretion, as described elsewhere herein. Administration, e.g., systemically, preferably orally, administering of the presently useful retinoid components, often occurs for more than about 1 week, preferably at least about 4 weeks, or at least about 6 weeks, or about 12 weeks or about 20 weeks. Daily doses of the retinoid component can vary over a wide range. In one embodiment, at least about 0.75 mg or at least about 1 mg or at least about 1.5 mg or at least about 3.0 mg or at least about 5 mg or about 6 mg or more of the retinoid component are administered to the human or animal on a daily basis. In one embodiment, the daily dose advantageously is in a range of about 1 mg to about 6 mg of the retinoid component.

In a very useful embodiment, the administering step comprises orally administering the retinoid. Although any means of oral administration may be used, the Applicants currently prefer the use of a capsule, for example, a hard gel capsule or a soft gel capsule, containing the retinoid component to the human or animal. Among the capsule systems useful in accordance with the present invention are those disclosed in Firestone et al., U.S. Pat. No. 6,248,354. Firestone et al. discloses capsules, including soft gel capsules with the following fill formulations:

| | | Concentration (mg/capsule) | |
|---|---|---|---|
| | | 0.7 mg Soft | 0.2 mg Soft |
| | | Gelatin | Gelatin |
| | | Capsule | Capsule |
| Ingredient | Function | (9096X) | (9154X) |
| Fill Formulation: | | | |
| Tazarotene | Active | 0.70 | 0.20 |
| Butylated | Anti-oxidant | 0.05 | 0.05 |
| Hydroxyanisole NF | | | |
| Sorbitan | Emulsifier | 5.0 | 5.0 |
| Monooleate NF | | | |
| Polysorbate 80 NF | Co-emulifier | 0.25 | 0.25 |
| Medium-chain | Lipophilic | 94.0 | 94.5 |
| Triglycerides EP | vehicle | | |

Of course, other formulations can be effectively used for oral administration of the presently useful retinoid components; in addition to hard gelatin capsules, tablets or powders (encapsulated or not) may be used. Also, the formulations including the presently useful retinoid components may be chosen or selected depending, for example, on the mode of systemic administration of the composition. For example, and without limitation, formulations for oral administration, transdermal administration, rectal (suppository) administration, administration by injection and other non-oral systemic administration advantageously have different chemical compositions one from the other. Accordingly, each formulation is made to provide the drug in a manner that suits the purpose and mode of administration, for which the drug is to be used. Different formulations for use in the same mode of administration may be employed, for example, to effectively or more effectively meet the needs and/or requirements of the patient and/or the application involved. For example, and without limitation, formulations for oral administration can be in forms including soft capsules, hard capsules, powders, pills, tablets, liquids, syrups, elixirs and the like and mixtures or combinations thereof.

Each and every feature described herein, and each and every combination of two or more of such features, is included within the scope of the present invention provided that the features included in such a combination are not mutually inconsistent.

These and other aspects and advantages of the present invention are set forth in the following detailed description, examples and claims.

### DETAILED DESCRIPTION

The present methods provide desired therapeutic effects employing certain retinoid components, particularly when administered systemically, for example, orally, to treat severe acne, for example nodular acne, cystic acne, and nodulocystic acne, in a manner that reduces the attenuation of sebum secretion at a dose that will provide substantially the same efficacy as a reference retinoid agent administered in the same manner.

In addition to the reduction on the effect on sebum seretion, other side effects commonly seen in other systemic retinoid drugs that can be reduced in severity or substantially eliminated in accordance with the present invention include, but are not limited to, metabolic and nutritional side effects, whole body side effects, endocrine side effects, hemic and lymphatic system side effects, digestive system side effects, ocular side effects, cardiovascular side effects, nervous system side effects, psychiatric side effects, typical retinoid toxicity side effects, respiratory system side effects, ear side effects, gastrointestinal tract side effects, and urinary system side effects.

The following are more specific examples of side effects which may be mitigated against in accordance with the present invention.
Typical Retinoid Toxicity: this side effect is similar to that in patients taking high doses of vitamin A and includes headache, fever, skin mucous membrane dryness, bone pain, nausea/vomiting, rash, mucositis, pruritus, increased sweating, visual disturbances, ocular disorders, alopecia, skin changes, changed visual acuity, bone inflammation, and visual field defects.
RA-APL Syndrome: characterized by fever, dyspnea, weight gain, radiographic pulmonary infiltrates and pleural or pericardial effusions. This syndrome is occasionally accompanied by impaired myocardial contractility and episodic hypotension and is observed with or without concomitant leukocytosis.
Body as a Whole: general disorders includes malaise, shivering, hemorrhage, infections, peripheral edema, pain, chest discomfort, edema, disseminated intravascular coagulation, weight increase, injection site reactions, anorexia, weight decrease, myalgia, flank pain, cellulitis, face edema, fluid imbalance, pallor, lymph disorders, acidosis, hypothermia, and ascites.
Respiratory System Disorders: include upper respiratory tract disorders, dyspnea, respiratory insufficiency, pleural effusion, pneumonia, rales, expiratory wheezing, lower respiratory tract disorders, pulmonary infiltration, bronchial asthma, pulmonary edema, larynx edema, and unspecified pulmonary disease.
Ear Disorders: ear disorders are consistently reported, with earache or feeling of fullness in the ears also reported. Hearing loss or other unspecified auricular disorders are observed, with infrequent reports of irreversible hearing loss. Gastrointestinal Tract (GI) Disorders: include GI hemorrhage, abdominal pain, other gastrointestinal tract disorders, diarrhea, constipation, dyspepsia, abdominal distention, hepatosplenomegaly, hepatitis, ulcer, and unspecified liver disorder.
Cardiovascular and Heart Rate and Rhythm Side Effects: arrhythmias, flushing, hypotension, hypertension, phlebitis, cardiac failure, cardiac arrest, myocardial infarction, enlarged heart, heart murmur, ischemia, stroke, myocarditis, pericarditis, pulmonary hypertension, and secondary cardiomyopathy.
Central and Peripheral Nervous System Disorders and Psychiatric Side Effects: dizziness, paresthesias, anxiety, insomnia, depression, confusion, cerebral hemorrhage, intracranial hypertension, agitation, hallucination, abnormal gait, agnosia, aphasia, asterixis, cerebellar edema, cerebellar disorders, convulsions, coma, CNS depression, dysarthria, encephalopathy, facial paralysis, hemiplegia, hyporeflexia, hypotaxia, no light reflex, neurologic reaction, spinal cord disorder, tremor, leg weakness, unconsciousness, dementia, forgetfulness, somnolence, and slow speech.
Urinary System Disorders: renal insufficiency, dysuria, acute renal failure, micturition frequency, renal tubular necrosis, and enlarged prostate.

In one useful embodiment, the systemically, preferably orally, administering step is effective to provide a maximum plasma concentration of an active retinoid agent in the human or animal of greater than about 30 ng/ml, preferably greater than about 40 ng/ml or 45 ng/ml or about 60 ng/ml or about 80 ng/ml, for example, greater than about 100 ng/ml. Of course, the concentration of active retinoid agent in the blood of the human or animal must be therapeutically effective and present in a concentration less than that amount which would cause substantial harm or toxicity in the patient.

Because of the reduction in the incidence and/or severity of side effects in accordance with the present methods, increased maximum blood concentrations of the presently useful retinoid components, relative to the maximum blood concentration of a reference retinoid agent, may be employed to the therapeutic advantage of the human or animal while still resulting in reduced risk of side effects. This is an important advantage of the present invention.

The concentration of the retinoid, including a tazarotene compound, in blood may be determined using a liquid chromatography-mass spectroscopy-mass spectroscopy (LC-MS/MS). In pharmaceutical applications, drug concentrations are typically reported in terms of blood plasma concentration rather than whole blood concentration. Thus, for the purposes of this application, references to “blood concentration” shall be understood to mean “blood plasma concentration.”

The systemically administering advantageously comprises other than topically administering the retinoid component (preferably the tazarotene compound, to the human or animal. Preferably, although not exclusively, the administering comprises a step selected from the group consisting of orally administering to the human or animal the retinoid component, transdermally administering to the human or animal the retinoid component, intravenously administering to the human or animal the retinoid component, subcutaneously administering to the human or animal the retinoid component, intramuscularly administering to the human or animal the retinoid component, intraperitoneally administering to the human or animal the retinoid component, rectally administering to the human or animal the retinoid component, one or more of like administering steps and combinations thereof. In a very useful embodiment, the administering comprises systemically, preferably orally, administering to the human or animal the retinoid component.

In a preferred embodiment the retinoid component is not topically administered to the skin of the human or animal in an amount effective to treat the patient's condition while, or during the time, the retinoid component is being systemically administered to the human or animal, for instance, to treat the same condition.

The retinoid component preferably includes a tarzarotene compound effective to selectively activate at least one of RAR-beta and RAR-gamma relative to RAR-alpha.

As used herein, the terms “selectively” or “more selectively” refer to the ability of an active retinoid agent to act as an agonist at RAR-beta and RAR-gamma relative to RAR-alpha. In preferred embodiments, the presently useful active retinoid agents are at least about 5 times more potent as an agonist of RAR-beta and RAR-gamma than RAR-alpha, or at least about 10 times more potent, or at least about 20 times more potent, or at least about 50 times more potent, or at least about 100 times more potent, or about 1000 times more potent than RAR-alpha. The term “specifically” refers to the ability of an active retinoid agent to affect RAR-beta and RAR-gamma at a therapeutically effective dose without detectably agonizing RAR alpha (to a statistically significant extent) using the assays disclosed in this specification.

In one embodiment, the retinoid component includes an active retinoid agent or a precursor of an active retinoid agent effective to selectively or even specifically affect both RAR-beta and RAR-gamma relative to RAR-alpha. The retinoid component advantageously includes an active retinoid agent or a precursor of an active retinoid agent effective to selectively or even specifically activate or inhibit the activation of or block at least one or both of RAR-beta and RAR-gamma relative to RAR-alpha. In one embodiment, the retinoid component includes an active retinoid agent or a precursor of an active retinoid agent effective to selectively or even specifically activate at least one of or both RAR-beta and RAR-gamma relative to RAR-alpha.

In a preferred embodiment the retinoid component comprises a tazarotene compound.

In one useful embodiment, the retinoid component includes, or upon systemic administration is able to be converted to, an active retinoid agent having a substantial degree of water solubility. For example, an active retinoid agent may be more water soluble than isotretinoin, or may be metabolically converted in the human or animal into an active retinoid agent having a substantial degree of water solubility. In this way, it is possible to design the active retinoid agent to avoid having the active agent cross lipid barriers, such as the blood brain barrier and the retinal-blood barrier.

Advantageously, the retinoid component comprises an active RAR ligand which is substantially ineffective to bind to or activate or block RXRs and/or a precursor of an active RAR ligand substantially ineffective to bind to or activate or block RXRs.

Preferably the retinoid compound is a tazarotene compound.

In a broad sense, any compound can be tested for RAR activity, for example, using conventional and well known techniques, for example, without limitation, those described in the above-noted patents, each of which is incorporated in its entirety herein by reference. Once a compound has been determined to have suitable RAR activity, it can be administered to a test animal with appropriate monitoring for drug interactions and/or side effects. Comparing the results of such monitoring with similar monitoring of test animals given reference retinoid agents allows one to determine if the compound is useful in accordance with the present invention.

In other aspects of the present invention, one or more compounds, for example, from a screening library of compounds, which are known to have or have been tested, using conventional and well known techniques, and found to have useful RAR activity, can be individually or collectively tested for RXR activity using conventional and well known testing procedures. See, for example, the above-noted Evans et al. patents, in particular U.S. Pat. No. 5,906,920, hereby incorporated by reference herein.

Further confirmation that a compound is useful in accordance with the present invention can be obtained by orally administering the compound to an animal and monitoring for therapeutic efficacy and/or the rate of sebum secretion.

In any event, determining which compounds are useful in accordance with the present invention can be accomplished using conventional and well known techniques, without undue experimentation.

Some examples of structures and methods of making preferred retinoid components, are provided in U.S. Pat. No. 5,776,699, U.S. Pat. No. 5,958,954, U.S. Pat. No. 5,877,207, and U.S. Pat. No. 5,919,970 which are all incorporated by reference herein in their entireties.

Such compounds can be made using well-known techniques. For example, see Klein et al U.S. Pat. No. 5,776,699, the disclosure of which has previously been incorporated in its entirety herein by reference. Therefore, a more detailed express description of the techniques or methods for working such compounds is not presented here.

The methods of the present invention are useful in the treatment of nodulocystic acne, for instance, severe nodulocystic acne, and are particularly beneficial because they result in less reduction, or even substantially no reduction, in sebum secretion. A reduction in sebum secretion often occurs upon use of other oral or systemic retinoid agents, resulting in itching, and dry, chapped skin.

Such use of retinoid components, particularly tazarotene compounds, in accordance with the present invention effectively provides treatment of nodulocystic acne without subjecting the patient to undue reduction in sebum secretion previously associated with treating nodulocystic acne with other retinoid active agents, for example, isotretinoin.

Especially useful retinoid components useful in the present methods include tazarotene, tazarotenic acid and mixtures thereof. Tazarotene is an ethyl ester prodrug that is metabolized to the corresponding free acid, tazarotenic acid. Tazarotene has a rigid ring-locked structure that offers limited conformational flexibility compared to all-trans-retinoic acid, the natural ligand for the retinoic acid receptors (RARs). This structural change confers tazarotenic acid with specificity for the RARs and selectivity for RAR-β and RAR-γ. As RAR-γ is the major receptor found in skin, tazarotene exerts its pharmacological effects through RAR-γ. Tazarotene is also a potent AP1 antagonist. AP1 regulates the transcription of many genes involved in proliferation and inflammation.

Tazarotenic acid does not activate the RXR retinoid receptors and its major metabolite, a sulfoxide derivative, does not activate either the RARs or the RXRs. As it has no isomerizable double bonds, tazarotene cannot be converted into RXR-active compounds.

By contrast, polyolefinic retinoids such as isoretinoin and acitretin can be isomerized, and if isomerization occurs metabolically the isomers could potentially activate the RARs and/or RXRs. RXR agonists cause transient elevation of triglycerides by inhibiting peripheral tissue lipoprotein lipase activity. RAR and RXR ligands act synergistically to induce hypertriglyceridemia. RAR pan agonists also induce hypertriglyceridemia by increasing hepatic triglyceride output, and this effect is primarily mediated by the RAR-α receptor. RAR-γ is not implicated in hypertriglyceridemia. As tazarotenic acid has minimal RAR-α activity and substantially no RXR activity, it would not be expected to elevate triglycerides—by either of the pathways.

Clinical use of RXR agonists has also been associated with hypothyroidism. As tazarotene is RAR specific, and cannot be either metabolized or isomerized to RXR active compounds, it would not be expected to cause either significant elevation of triglycerides or hypothyroidism.

The substantial absence of RXR activity and the minimal RAR-α a activity of tazarotenic acid are important factors that reduce the potential for some toxicities, such as hypertriglyceridemia and hypothyroidism, that are typically associated with oral or systemic retinoids.

The LC-MS/MS test for simultaneous detection of tazarotene and tazarotenic acid may be run as follows. One ml of plasma (EDTA-treated) is diluted with 1.0 ml of water. Diluted plasma is extracted using solid phase extraction (SPE) on a C18 cartridge. The eluate is evaporated, reconstituted in a water/methanol-based mobile phase, and injected onto a 4.6×50 mm, 3 μm pore size C-8 reverse phase high pressure liquid chromatography (HPLC) column (Agilent, Wilmington, Del.). Compounds are gradient-eluted at 1.2 mL/min and detected using an API 3000 triple quadrupole mass spectrometer with an Atmospheric Pressure Chemical Ionization (APCI) source (PE-Sciex, Concord, Ontario, Canada). Molecular reaction monitoring enhances the sensitivity and selectivity of this assay by collisionally dissociating the protonated molecules for the analyte and an internal standard thereby forming the product ions. The specific precursor-product ion pair monitored are m/z 352→324 for tazarotene, m/z 359→331 for the tazarotene internal standard, m/z 324→294 for tazarotenic acid, and m/z 331→298 for the tazarotenic acid internal standard. The lower limit of quantitation at assay range tested is 0.1 ng/mL, with a coefficient of variation and deviation from nominal concentration of <15%.

Retinoid components useful in the present invention may be included in a composition with one or more other suitable pharmaceutically acceptable ingredients. Examples of useful other ingredients include, but are not limited to antioxidants, such as burylated hydroxyanisole NF and the like; emulsifiers, such as sorbitanmonoolate NF, polysorbate 80 NF and the like and mixtures thereof; vehicle components, such as conventional vehicles and the like; and other materials which are useful to provide one or more benefits to the composition to be administered and/or to the subject to whom the composition is administered.

Additionally, the retinoid component may be coadministered, either in separate composition or together in a single composition, with one or more active ingredient. For example, tazarotene compounds may be coadministered with an antibiotic or other agent effective for the treatment of nodulocystic acne.

Daily dosages of the presently useful retinoid components may vary from patient to patient depending, for example, on the specific activity of the particular compound, on the desired therapeutic effect to be achieved, on the condition of the patient, on the mode of systemic administration, on the frequency of administration and the like factors. Such dosages advantageously are selected to provide the desired therapeutic effect, preferably substantially without unduly harming or interfering with the patient. Examples, without limitation, of such daily dosages may be in a range of about 0.1 mg/day or less or about 0.3 mg/day to about 7 mg/day or about 10 mg/day or more. Each oral dose may contain about 0.2 mg, or about 0.7 mg, or about 1 mg, or about 4.5 mg.

When the desired therapeutic effect is a reduction in nodulocystic acne, for example, severe nodulocystic acne, daily dosages are often within +the above-noted ranges. When tazarotene is orally administered to effect a reduction in such acne, the daily dosage of tazarotene preferably is in a range of about 0.3 mg/day to about 7 mg/day or about 8 mg/day, more preferably in a range of about 0.6 mg/day to about 6.5 mg/day or about 7 mg/day. Clinical trials using orally administered tazarotene to effect reductions, as described elsewhere herein, in nodulocystic acne have employed daily dosages of tazarotene including 0.4 mg/day, 0.75 mg/day, 1.5 mg/day, 2.8 mg/day, 3 mg/day, 4.5 mg/day, 6 mg/day and 6.3 mg/day. Each oral dose may contain about 0.4 mg, or about 0.75 mg, or about 1.5 mg, or about 2.8 mg, or about 3 mg or about 6 mg of tazarotene.

Although the presently useful retinoid components can be advantageously administered on a once daily basis, other dosing frequencies may be employed. For example, the presently useful retinoid components may be administered twice or three or more times daily, or once every two or three or more days.

The following non-limiting examples illustrate certain aspects of the present invention.

### EXAMPLE 1

Coadministration of 6.3 mg oral tazarotene with a high-fat meal in normal healthy subjects following single and multiple dose administrations does not substantially affect the bioavailability or pharmacokinetics of tazarotenic acid, the primary active retinoid species in the systemic circulation. This result is based on comparing the pharmacokinetics of tazarotenic acid when administered within 30 minutes after consuming a high fat breakfast vs. when administered after an 8-10 hour fast. The 90% confidence intervals (CI) of AUC ratios (test/reference) are completely within the 80-125% boundary. The 90% CI ratio of Cₘₐₓ values are partially outside the 80-125% boundary due to data variability, but the average ratios of 1.00 (Day 0) and 0.829 (Day 9) are within the above-noted limit.

Each of isotretinoin, acitretin and bexarotene is commercially available as an oral active retinoid agent. To one degree or another, patients taking each of these agents are instructed to take the agent with food to achieve improved drug absorption or bioavailability. This substantial dependence on the presence of food to achieve improved bioavailability clearly distinguishes these agents from the retinoid components useful in the present invention.

### EXAMPLE 2

A series of Phase 3 studies are conducted on orally administered tazarotene for the treatment of psoriasis. Adverse events (side effects) are monitored. Results of such monitoring are shown in Table 1. In addition, published data for acitretin's side effects are also considered. Table 1 shows the adverse events reported for at least 10% of the patients in the studies of acitretin versus those seen with tazarotene.

TABLE 1

| | NUMBER OF (%) OF PATIENTS | | | |
|---|---|---|---|---|
| | | Taz 4.5 mg | Taz 4.5 mg | |
| | | Study 3 | Study 3 | |
| | Taz 4.5 mg | treated | treated | |
| | Combined | with | with | |
| | data from | Tazarotene | Tazarotene | |
| | Studies 1 | for 6 | for 3 | |
| | and 2 | months | months | Acitretin |
| Adverse Event | (N = 348) | (N = 92) | (N = 220) | (N = 525) |
| Cheilitis | 228 (65.5) | 65 (70.7) | 149 (67.7) | 429 (81.7) |
| Skin | 3 (0.9) | 1 (1.1) | 3 (1.4) | 345 (65.7) |
| peeling/ | | | | |
| Desquamation | | | | |
| Alopecia | 1 (0.3) | 5 (5.4) | 1 (0.5) | 319 (60.8) |
| Dry Skin | 82 (23.6) | 24 (26.1) | 47 (21.4) | 174 (33.1) |
| Nail Disorder | 2 (0.6) | 1 (1.1) | 1 (0.5) | 170 (32.4) |
| Pruritus | 21 (6.0) | 3 (3.3) | 11 (5.0) | 157 (29.9) |
| Rhinitis | 8 (2.3) | 0 (0.0) | 0 (0.0) | 153 (29.1) |
| Sticky skin/Skin | 1 (0.3) | 0 (0.0) | 0 (0.0) | 129 (24.6) |
| disorder | | | | |
| Xerophthalmia/ | 8 (2.3) | 1 (1.1) | 3 (1.4) | 112 (21.3) |
| Eye disorder | | | | |
| Arthralgia | 60 (17.2) | 30 (32.6) | 28 (12.7) | 102 (19.4) |
| Dry mouth/Oral | 11 (3.2) | 2 (2.2) | 11 (5.0) | 76 (14.5) |
| dryness | | | | |
| Rigors/Chills | 9 (2.6) | 1 (1.1) | 1 (0.5) | 67 (12.8) |
| Hyperesthesia | 0 (0.0) | 0 (0.0) | 0 (0.0) | 66 (12.6) |
| Paronychia/Nail | 2 (0.6) | 1 (1.1) | 1 (0.5) | 66 (12.6) |
| disorder | | | | |
| Atrophy of skin | 0 (0.0) | 0 (0.0) | 0 (0.0) | 64 (12.2) |
| Epistaxis | 2 (0.6) | 0 (0.0) | 1 (0.5) | 61 (11.6) |
| Erythematous | 2 (0.6) | 0 (0.0) | 0 (0.0) | 57 (10.9) |
| rash/ | | | | |
| Erythema | | | | |
| Paresthesia | 7 (2.0) | 1 (1.1) | 3 (1.4) | 56 (10.7) |
| Back pain | 16 (4.6) | 10 (10.9) | 7 (3.2) | 41 (7.8) |

The results shown in Table 1 demonstrate that tazarotene compounds, when delivered systemically in a therapeutically effective amount, results in substantial reductions in, and even in some cases elimination of, side effects relative to the side effects resulting from the administration of acitretin.

### EXAMPLE 3

Two multicenter, double-blind, randomized, placebo-controlled 24-week studies of identical design are conducted to evaluate the safety of oral tazarotene. In addition, 16- to 24-week dose-response evaluations are performed.

In the two safety trials, the incidence of adverse side effects with a 4.5 mg dose administered orally once daily is compared to the incidence of the same side effects with a placebo. The following side effects are found to occur significantly more frequently with tazarotene than with the placebo: cheilitis (66% vs 17%), dry skin (24% vs 15%), headache (19% vs 12%), arthralgia (17% VS 8%), myalgia (14% vs 8%), back pain (7% vs 3%), joint disorder (4% vs 1%), nasal dryness (4% vs 1%), foot pain (3% vs 1%), rash (3% vs 1%), hyperglycemia (2% vs 0%), and dermatitis (1% vs 0%). The majority of these were mild in severity and typical of adverse effects associated with oral retinoids. Particularly noteworthy is that other adverse effects typically associated with oral retinoids—including hypertriglyceridemia, hypercholesterolemia, abnormal liver function tests, increased alanine aminotransferase, increased aspartate aminotransferase, desquamation, eye dryness, and alopecia—occur with essentially the same incidence with oral tazarotene as with placebo.

There are no statistically significant between-group differences in the incidence of ocular, auditory, or thyroid problems. Altered hormone levels (which includes elevated TSH and T4, decreased T4, and abnormal thyroid function test) occur significantly more frequently with placebo (2%) than with tazarotene (0%).

There is also no evidence that tazarotene was associated with an increased incidence of psychiatric disorders—depression (1% with tazarotene vs 2% with placebo), psychosis (0% vs <1%), psychotic depression (0% vs <1%), emotional lability (3% vs 3%), anxiety (1% vs <1%), and agitation (<1% vs 0%).

Data from the two identical trials plus the dose-response evaluations show that the incidence of patients discontinuing due to adverse effects is approximately 2% with 0.4 to 1.1 mg oral tazarotene (n=105), 10% with 2.1 to 2.8 mg oral tazarotene (n=21), 0% with w.2 mg (n=14), 3% with 4.5 mg (n=348), 13% with 6.3 mg (n=16), and 3% with placebo (n=383).

The results show that oral tazarotene appears to have safety and tolerability advantages over many other systemic treatments.

### EXAMPLE 4

Two placebo-controlled dose-ranging studies are conducted to evaluate the safety of oral tazarotene in patients having nodulocystic acne. In the first study, patients receive orally administered tazarotene at daily doses of 0.4 mg to 2.8 mg in 96 (71+25) patients (12 weeks treatment plus 12 weeks post-treatment). In the second study, daily doses of 0.75 mg to 6 mg tazarotene are administered to 181 patients (24 weeks treatment plus 12 weeks post-treatment).

Oral tazarotene is well tolerated, with only 2.5% (7/277) of patients withdrawing from either study due to adverse events (2 each with placebo, 0.75 mg, and 3 mg, and 1 with 6 mg).

The most common adverse events occurring during the treatment period in the placebo groups combined (n=54) or the three highest tazarotene dose groups (2.8 mg, n=11; 3 mg, n=37; and 6 mg, n=36) include cheilitis (31%, 64%, 78%, and 94%, respectively), dry skin (19%, 18%, 35%, 50%), headache (28%, 9%, 19%, 36%), arthralgia (6%, 9%, 8%, 28%), myalgia (9%, 0%, 16%, 25%), joint disorder (0%, 27%, 14%, 19%), and asthenia (13%, 0%, 19%, 19%). These events are predominantly mild or moderate in severity. For example, in the 3 mg and 6 mg groups, cheilitis is mild in 25 and 27 patients, respectively, moderate in 3 and 6 patients, and severe in 1 patient in each group. In the same groups, dry skin is mild in 11 and 17 patients, respectively, and moderate in 2 and 1 patient. No patient has severe dry skin.

Emotional lability occurs in 1 (3%) patient in the 3 mg group and 5 (14%) in the 6 mg group compared with 2 (4%) with placebo. All cases of emotional lability are mild. Depression occurs in 3 (8%) patients in the 3 mg group (2 mild, 1 severe), none in the 6 mg group, and 1 (2%)(moderate) with placebo.

There are no consistent dose-related clinically significant changes in urinalysis, chemistry, or hematology measures (including the results of liver function tests and levels of triglycerides, total cholesterol, and HDL cholesterol). Tazarotene treatment is also not associated with any clinically significant ligament calcification, osteophyte formation, or changes in serum bone alkaline phosphatase, serum amino terminal telopeptides, or bone density.

The results suggest that oral tazarotene has a good safety and tolerability profile in the treatment of nodulocystic acne and does not appear to result in clinically significant changes in liver enzymes, cholesterol or triglyceride levels, or bone density.

### EXAMPLE 6

Studies are conducted on orally administered tazarotene for the treatment of acne. Adverse events (side effects) are monitored.

Results of such monitoring are shown in Table 2. Published data for isotretinoin's side effects are also considered. Table 2 shows the adverse events reported for at least 10% of the patients in the studies of isotretinoin versus those for tazarotene.

TABLE 2

NUMBER OF (%) OF PATIENTS

| | Tazarotene | |
|---|---|---|
| | (N = 223) | |
| | Combined data | |
| | from Studies 1 | Isotretinoin |
| Adverse Event | and 2 | (N = 525) |
| Cheilitis | 134 (60.1) | 472 (90.2) |
| Conjunctivitis | 0 (0.0) | 201 (38.4) |
| Desquamation central | 1 (0.4) | 187 (35.8) |
| face/Desquamation | | |
| Dry skin | 79 (35.4) | 172 (32.9) |
| Skin fragility/Skin | 1 (0.4) | 164 (31.4) |
| disorder | | |
| Dry nose | 7 (3.1) | 136 (26.0) |
| Pruritus | 10 (4.5) | 127 (24.3) |
| Epistaxis | 6 (2.7) | 121 (23.1) |
| Peeling/Desquamation | 1 (0.4) | 109 (20.8) |
| Irritation of eyes | 0 (0.0) | 85 (16.3) |
| Rash-dermatitis/Rash | 7 (3.1) | 82 (15.7) |
| Fingertip | 1 (0.4) | 63 (12.0) |
| peeling/Desquamation | | |
| Joint pain/Arthralgia | 18 (8.1) | 60 (11.5) |
| Red Scaly | 1 (0.4) | 54 (10.3) |
| face/Desquamation | | |
| Headache | 26 (11.7) | 44 (8.4) |

The results shown in Table 2 demonstrate that tazarotene (in a therapeutically effective amount) when used to treat acne in accordance with the present invention results in substantial reductions in, and even in some cases elimination of, side effects relative to the side effects resulting from the administration of isotretinoin.

### EXAMPLE 7

A multicenter, double-blind, randomized, placebo-controlled parallel-group study is undertaken to determine the efficacy of orally administered tazarotene in treating severe nodulocystic acne.

The main inclusion criteria for this study include: at least 7 facial nodulocystic acne lesions (>5 mm); an age of at least 16 years; stable doses of any concurrent medication that might significantly affect hepatic or renal excretion; if taking oral contraceptives, stable dose for last 3 months; and negative urine pregnancy test for females of childbearing potential.

The main exclusion criteria for this study include: females of childbearing potential not committed to using highly effective contraceptive during the study; pregnant or lactating females; 8-hour fasting triglyceride levels ≧500 mg/dL, serum calcium levels >11 mg/dL; likelihood of prolonged exposure to ultraviolet light during the study; and uncontrolled systemic disease.

In addition, washout periods for other medications for this study are: 1 week for vitamin A supplements >5000 IU; 2 weeks for topical anti-acne medications (e.g., retinoids, azelaic acid, benzoyl peroxide); 2 weeks for topical or systemic antibiotic therapy that may alter the course of acne; and 6 months for systemic retinoids.

Treatment Regimen

Patients are randomized to receive placebo or oral tazarotene (0.75, 1.5, 3, or 6 mg), in a 1:1:1:1:1 ratio once daily for 24 weeks. After this, the patients are followed without treatment for an additional 12 weeks. Patients discontinuing from the treatment period due to adverse effects or lack of efficacy are eligible for entry into the post-treatment phase.

Main Outcome Measures

In the study, overall acne severity is rated as: none (no inflammatory acne lesions); mild (few to several papules or pustules, no nodulocystic lesions); moderate (several to many papules or pustules, few to several nodulocystic lesions); and severe (numerous or extensive papules or pustules, many, for example, at least about 5 or at least about 10, nodulocystic lesions).

Treatment success is defined as at least moderate (about 50%) improvement on the following 7-point global response scale:
0=completely cleared
1=almost cleared (about 90% improvement)
2=marked response (about 75% improvement)
3=moderate response (about 50% improvement)
4=slight response (about 25% improvement)
5=condition unchanged
6=condition worsened

In this study, facial nodulocystic lesion count includes lesions greater than 5 mm in size. Facial papular/pustular lesion count includes lesions less than or equal to 5 mm in size. Facial non-inflammatory lesion count includes open and closed comedones.

Other Measures

Sebum output is assessed every 4 weeks at selected centers using the Sebutape® patches, sold by Cuderm Corporation. Urinalysis, chemistry, and hematology values are monitored. Bone formation and resorption assessments (serum bone alkaline phosphatase and serum amino terminal telopeptides, respectively) at selected centers are monitored.

Bone mineral density of spine and proximal femur at selected centers is monitored. Ligament calcification or osteophyte formation (lateral X-ray of the cervical and thoracic spine, and ankle calcaneous) is monitored. Epiphyseal growth plate closure (internal oblique X-ray of the ankle in patients less than or equal to 21 years old) is monitored.

Results

Patients

181 patients enroll in the study. 127 (70%) of the patients complete the 24-week treatment phase. 145 patients enter the 12-week post-treatment phase. 96 (66%) of these patients complete this phase. The study population is nearly equally divided between males and females (55% males) and is ethnically diverse (61% Caucasian, 22% Hispanic, 12% black, 4% Asian, 1% other). The mean age is 22.7 years. The mean number of facial nodulocystic lesions at baseline ranges from 10.8 to 12.2 in the treatment groups. There are no significant differences between the groups at baseline in demographics or measures of acne severity.

In the treatment period, few patients withdraw due to adverse events that are unrelated to or possibly or definitely related to treatment. The withdrawing patients are as follows: 0% (0/36) of placebo group; 6% (2/35) of 0.75 mg group (anxiety attack, mild leucopenia); 0% (0/37) of 1.5 mg group; 5% (2/37) of 3 mg group (infectious mononucleosis, depression); and 3% (1/36) of 6 mg group (spinal stiffness and joint and muscle pain).

Patients withdrawing due to lack of efficacy are primarily in the placebo or lowest dose groups: 17% (6/36) of placebo group; 20% (7/35) of 0.75 mg group; 5% (2/37) of 1.5 mg group; 0% (0/37) of 3 mg group; and 6% (2/36) of 6 mg group.

Main Outcome Measures

Results of this study are that tazarotene at 6 mg reduces the overall acne severity significantly more than placebo from week 16 until the end of the post-treatment phase (p≦0.01). More than 45% of the patients in the three highest dose groups have either no acne or mild acne by the end of the treatment phase, compared with 34% in the tazarotene 0.75 mg group and 19% in the placebo group. At the end of the post-treatment phase, these levels of acne have been maintained in 53% of the 6 mg group and 43% of the 3 mg group.

Tazarotene at 3 mg and 6 mg have a significantly higher incidence of treatment success (≧50% global improvement) than placebo at week 24 and throughout the post-treatment phase (p≦0.05). Treatment success is achieved by week 12 in more than 70% of patients treated with the three highest doses of tazarotene and by week 24 in more than 86% of patients treated with the two highest doses. Tazarotene achieves consistently greater reductions in the number of total facial nodulocystic lesions than placebo from week 8 onward. At the end of the treatment period, the mean total facial nodulocystic lesion count is reduced from:
11.6 to 5.2 in the placebo group (a 55% reduction);
12.2 to 4.2 in the 0.75 mg group (a 66% reduction);
11.8 to 3.2 in the 1.5 mg group (a 73% reduction);
10.8 to 2.3 in the 3 mg group (a 79% reduction); and
11.6 to 1.6 in the 6 mg group (an 86% reduction).

The percentage of patients with at least a 90% reduction in facial nodulocystic lesion count is significantly greater in the higher-dose tazarotene groups (1.5, 3, and 6 mg) than in the placebo group at week 24. The 6 mg group also shows significant superiority over placebo at the end of the post-treatment phase. The median time to initial complete clearing of facial nodulocystic lesions is less in the 3 mg and 6 mg groups (16 weeks in both groups) than in the placebo group (24 weeks) (p=0.017 and p=0.081, respectively).

From week 8 onward, tazarotene results in consistently greater reductions in the number of facial papules or pustules, and facial non-inflammatory acne lesions, compared with placebo. At the end of the treatment period, the mean facial papule or pastule count is reduced from:
32.4 to 22.3 in the placebo group (a 31% reduction);
32.3 to 20.3 in the 0.75 mg group (a 37% reduction);
29.1 to 13.3 in the 1.5 mg group (a 54% reduction);
25.4 to 10.0 in the 3 mg group (a 61% reduction); and
24.6 to 11.1 in the 6 mg group (a 55% reduction).

At the end of the treatment period, the mean facial non-inflammatory lesion count is reduced from:
62.3 to 34.2 in the placebo group (a 45% reduction);
56.3 to 30.3 to 30.3 in the 0.75 mg group (a 46% reduction);
59.2 to 17.8 in the 1.5 mg group (a 70% reduction); 56.1 to 21.3 in the 3 mg group (a 62% reduction); and 47.7 to 13.5 in the 6 mg group (a 72% reduction).

Sebum secretion output is assessed in a maximum of 86 patients (with successively fewer patients at each timepoint—e.g., 60 patients at week 24, 46 patients at week 36). Importantly, there are no consistent statistically significant differences in sebum production across treatment groups using the Sebutape® method of assessment. In other words, the oral administration of tazarotene does not substantially reduce sebum secretion relative to placebo, even when such tazarotene administration is effective to reduce or even eliminate severe nodulocystic acne.

The most common adverse events (i.e., with an incidence of ≧15%) occurring in the treatment period are cheilitis, dry skin, arthralgia, and joint disorder. For each of these adverse events, the majority of cases are mild.

None of the treatment groups show any consistent clinically significant changes in urinalysis, chemistry, or hematology measures (including liver function test results and levels of triglycerides, total cholesterol, and high-density lipoprotein cholesterol).

Bone formation and resorption do not appear to be altered. There are no statistically significant differences between groups in the mean change from baseline in serum bone alkaline phosphatase (bone formation) or serum amino terminal telopeptides (bone resorption).

None of the treatment groups show any clinically significant ligament calcification, osteophyte formation, or changes in bone density. All patients have distal tibial growth plate closure at both day 0 and the last follow-up visit. Two patients have physes that are partially closed at baseline and both close completely during the study in an unremarkable fashion.

The results of this study suggest that oral tazarotene has a better tolerability profile than other oral retinoids. Oral isotretinoin has been associated with several adverse events that did not occur as frequently—or at all—with oral tazarotene. For example, the only adverse effects reported with an incidence of ≧15% with oral tazarotene are cheilitis, dry skin, headache, arthralgia, myalgia, infection, asthenia, and joint disorder. Furthermore, oral tazarotene is not generally associated with abnormalities in liver function test results or elevated levels of triglycerides, total cholesterol, or high-density lipoprotein cholesterol.

Oral tazarotene is efficacious at once-daily doses of 1.5, 3, and 6 mg. The higher doses are associated with the greatest efficacy, the most rapid clearing of facial nodulocystic lesions, and maintenance of response for at least 12 weeks post-treatment. The superiority of the 6-mg dose of oral tazarotene over placebo is significant from week 16 onward. As noted above, oral administration of tazarotene does not substantially reduce sebum secretion, even when such administration is effective to reduce or even eliminate severe nodulocystic acne.

While this invention has been described with respect to various specific examples and embodiments, it is to be understood that the invention is not limited thereto and that it can be variously practiced within the scope of the following claims:

1. A method of treating severe nodulcystic acne in an animal or human comprising administering a therapeutically effective systemic dose of a composition comprising a tazarotene compound.

2. The method of claim 1 wherein said composition is administered orally.

3. The method of claim 2 wherein said composition comprises a solid dosage form.

4. The method of claim 3 wherein said solid dosage form is selected from the group consisting of a tablet, a powder, and a capsule.

5. The method of claim 4 wherein said solid dosage form comprises a capsule.

6. The method of claim 5 wherein said capsule comprises a hard gelatin capsule.

7. The method of claim 5 wherein said capsule comprises a soft gelatin capsule.

8. The method of claim 1 wherein said systemic dose is a administered transdermally.

9. The method of claim 1 wherein said systemic dose is administered rectally.

10. The method of claim 1 wherein said systemic dose is administered intravenously.

11. The method of claim 1 wherein said systemic dose is administered intraperitoneally.

12. The method of claim 1 wherein said tazarotene compound is selected from the group consisting of tazarotene and tazarotenic acid.

13. The method of claim 12 wherein said tazarotene compound is tazarotene.

14. The method of claim 13 wherein said composition is administered orally.

15. The method of claim 14 wherein said composition comprises a solid dosage form.

16. The method of claim 15 wherein said solid dosage form is selected from the group consisting of a tablet, a powder, and a capsule.

17. The method of claim 16 wherein said solid dosage form comprises a capsule.

18. The method of claim 17 wherein said capsule comprises a hard gelatin capsule.

19. The method of claim 17 wherein said capsule comprises a soft gelatin capsule.

20. The method of claim 13 wherein said systemic dose is a administered transdermally.

21. The method of claim 14 wherein said systemic dose is administered rectally.

22. The method of claim 15 wherein said systemic dose is administered intravenously.

23. The method of claim 16 wherein said systemic dose is administered intraperitoneally.

24. The method of claim 12 wherein said tazarotene compound is tazarotenic acid.

25. A method for the treatment of severe nodulocystic acne in a human or animal comprising administering to said human or animal a therapeutically effective dose of a tazarotene compound.

26. The method of claim 25 wherein said tazarotene compound is tazarotenic acid.

27. The method of claim 26 wherein said tazarotenic acid is administered in a systemic dosage form.

28. The method of claim 26 wherein said tazarotenic acid is formed in the gastrointestinal tract of said human or mammal.

29. The method of claim 26 wherein said tazarotenic acid is formed in the blood of said human or mammal.

30. The method of claim 25 wherein said tazarotene compound is tazarotene.

31. The method of claim 30 wherein said tazarotene is administered to said animal or human in a systemic dosage form.

32. The method of claim 31 wherein said systemic dosage form is an oral dosage form.

33. The method of claim 31 wherein said oral dosage form is selected from the group consisting of a tablet, a powder, and a capsule.

34. The method of claim 33 wherein said solid dosage form comprises a capsule.

35. The method of claim 34 wherein said capsule comprises a hard gelatin capsule.

36. The method of claim 34 wherein said capsule comprises a soft gelatin capsule.

37. The method of claim 31 wherein said systemic dose is a administered transdermally.

38. The method of claim 31 wherein said systemic dose is administered rectally.

39. The method of claim 31 wherein said systemic dose is administered intravenously.

40. The method of claim 16 wherein said systemic dose is administered intraperitoneally.

41. A method of treating severe nodulocystic acne in an animal or human comprising administering to said human or animal an therapeutically effective systemic dose of tazarotene, wherein said dose produces at least one fewer or less severe side effect than a systemic dose having similar efficacy of isotretinoin.

42. A method of treating severe nodulcystic acne in an animal or human comprising administering to said human or animal an oral dose of tazarotene of about 4.5 mg/day.
